(19) United States
(12) Patent Application Publication
(10) Pub. No.: US 2015/0216797 A1
(43) Pub. Date: Aug. 6, 2015

(54) METHOD FOR SATIATION ENHANCEMENT OF FOOD EMULSIONS USING AN AEROSOL DELIVERY SYSTEM

(71) Applicant: Jeffrey Fergus Wilkinson, Sacramento, CA (US)

(72) Inventor: Jeffrey Fergus Wilkinson, Sacramento, CA (US)

(21) Appl. No.: 14/170,424

(22) Filed: Jan. 31, 2014

(51) Int. Cl. A61K 9/00; A61K 9/12

(52) U.S. Cl. CPC A61K 9/0056; A61K 9/12

(57) ABSTRACT

The method as described herein promotes reduced food calorie intake, for purposes of weight loss or weight maintenance, through use of an aerosol delivery system that increases food emulsion volume (overrun) by as much as 400%, thereby stimulating various satiation feedback mechanisms within the body to effectively gain a net increase in satiation when compared to satiation from calories otherwise ingested without the volume increase produced by said aerosol delivery system.

### REFERENCE TO SEQUENCE LISTING, A TABLE, OR A COMPUTER PROGRAM LISTING COMPACT DISK APPENDIX

Not Applicable

### BACKGROUND OF THE INVENTION

The present invention is in the technical field of human physiology. More particularly, the present invention is in the technical field of hunger satiation and appetite regulation. More particularly, the present invention is in the technical field of weight loss and/or weight management.

An unprecedented availability of inexpensive food combined with a food marketing/manufacturing philosophy that encourages the creation of inexpensive and physiologically irresistible processed foods has led to a pervasive inability of most Americans to control their calorie intake. To make matters worse, food manufacturers, by and large, have little vested interest in the nutritional quality or healthiness of the foods they are selling. In fact, it is often to their advantage to create foods with a low satiation index so that consumers will eat more of their product.

According to the Center for Disease Control, nearly two-thirds of American adults are overweight or obese. Obesity with children and young adults has also been increasing at an alarming rate for the last two decades.

Obesity, the #2 cause of preventable death in the US, is having a devastating effect on our healthcare system and overall US productivity. The total economic cost associated with America's weight crisis was estimated to be $270 billion in 2011 alone. Studies confirm that many deadly diseases like type 2 diabetes, hypertension, heart disease, stroke, kidney and liver disease and certain cancers are related to unhealthy eating and obesity.

Despite spending approximately $30 billion each year on weight loss programs, plans, services, supplements, drugs, devices and foods, Americans have found little success in shedding and keeping off the excess weight.

The tremendous demand for a solution to unwanted weight gain has spawned myriad inventions, from diet pills to diet shakes. Some examples of prior art that presently appear more relevant are discussed below:

Diet Pills:

Diet pills, both prescription and over the counter, have been around since the 1950's and use stimulants of varying degrees and toxicities to curb appetite and speed metabolism with weight loss as the main objective. According to the FDA, long term use of stimulants may lead to heart damage, strokes, and kidney failure.

Herbal Diet Products:

Various diet food and drug manufacturers have promoted myriad herbs as having appetite suppressing qualities. The use of many of these herbs and formulations have received warnings from the FDA regarding their safety and efficacy. Currently, there are no herbs on the weight loss market that have been proven to be both safe and effective for long term use.

Diet Drinks/Foods:

With the advent of artificial sweeteners, food manufacturers have developed a cadre of low calorie or no calorie foods. Artificial sweeteners have many critics who question their safety and efficacy and some have been proven to cause cancer and other health risks in animal studies. One study from Division of Epidemiology, University of Texas Health Sciences Center, concluded that, “daily consumption of diet soda was associated with a 36% greater relative risk of incident metabolic syndrome and a 67% greater relative risk of incident type 2 diabetes compared with nonconsumption.” Studies suggest that continued use of artificial sweeteners alters the body's ability to regulate calorie intake by disrupting satiation feedback mechanisms.

Weight Loss Shakes/Emulsions:

Liquid emulsions and powders (intended for rehydration) have a long history as convenient meal replacements or controlled calorie snacks. Many of these products include protein, vitamin and mineral fortification. Although these products have stood the test of time (unlike most diet concepts), they work on the most basic satiation principles and offer little net satiation beyond the calories consumed. Their effectiveness seems to depend more on portion/calorie control and the dieter's will power than on their ability to provide meaningful satiation (hunger relief) beyond their calorie content.

Tastants/Olfactory Inhalants:

Studies suggest that adding tastants to food, or using olfactory scent inhalants prior to meals engages satiation feedback mechanisms in the palette and olfactory sensors which help in appetite regulation. Although there is support in the scientific literature for improved satiation from tastants/olfactory inhalants, they do not take advantage of several of the body's other satiation feedback mechanisms.

### SUMMARY OF THE INVENTION

The present invention is a method to promote appetite regulation by transforming nutritionally, functionally and calorically tailored food emulsions into semisolids, with volume expanded by as much as 400%, thereby stimulating hunger satiation responses, said volume expansion being achieved through use of an aerosol delivery system.

### DETAILED DESCRIPTION OF THE INVENTION

In the current application, the term “satiation” is generally understood to mean the human body's immediate reaction to the ingestion of food—the drive that causes a person to stop eating. It is the body's attempt to estimate future satiety via sensory input: smell, taste, texture, and stomach distention.

In the current application, the term “satiation feedback mechanisms” is generally understood to mean the various signals resulting from the ingestion of energy that feed into specific areas of the brain involved in the regulation of energy intake, in response to the sensory and cognitive perceptions of the food or drink consumed, and distension of the stomach. These signals are integrated by the brain, and satiation is stimulated. Nutrition Bulletin Volume 34, Issue 2, pages 126-173, June 2009 Satiation, satiety and their effects on eating behavior, B. Benelam

In the current application, the term “semisolid” is generally understood to mean the product of the invention once dispensed from the aerosol delivery system.

For purposes of this application, the term “foam” is generally understood to mean the product of a food emulsion with the characteristics to produce food foam once sufficiently whipped, agitated or dispensed from an aerosol delivery system.

In the current application, the terms “transformed”, “transforming” or “transformation” are generally understood to mean the conversion of a food emulsion into a semisolid form.

In the current application, the term “stable foam” or “foam stability” is generally understood to mean foam that maintains at least 70% of its volume for at least 30 seconds after being dispensed from the aerosol delivery system.

Now referring to the invention in more detail:

### Component 1—Aerosol Delivery System:

One embodiment of the aerosol delivery system consists of a propellant, such as nitrous oxide gas, and a food emulsion, both of which are filled, observing the appropriate sequence, into a disposable aerosol package, typically a metal can, plastic nozzle mechanism and cap designed for use in aerosol whipped topping production. This process of transforming emulsions into semisolids is well known to those skilled in the art of aerosol whipped topping manufacture. In one embodiment, the delivery of said semisolid is intended to be from the express point of the nozzle directly into the mouth of a person for ingestion. For the emulsion to be successfully transformed and dispensed from an aerosol package—an aerosol can in this embodiment—the can should be inverted so that the bottom of the can is above the nozzle by at least 10 degrees at the time the nozzle is depressed and the semisolid dispensed. Bacterial contamination is avoided by consuming the contents of the aerosol package in one sitting, as with embodiments designed for single serve capacity, or by cleansing the nozzle between servings for embodiments with larger capacities. Inadvertent inhalation of nitrous oxide would be negligible even when instructions are not properly followed by the consumer.

Another embodiment of the aerosol delivery system consists of a propellant, such as nitrous oxide gas, and a food emulsion, both of which are filled, observing the appropriate sequence, into a reusable canister commonly available for home and commercial use designed for the purpose of converting emulsions like heavy whipping cream into aerosol foam.

### Component 2—Food Emulsion:

A necessary characteristic for food emulsions to be transformed into semisolids through use of an aerosol delivery system is that they have foaming properties, which are generally determined by either their fat or protein content as the bubble structure of food foams is generally comprised of fat, protein or a combination of the two. Fat based foam, like whipped heavy cream, is characterized by its foam stability, silky mouth feel, and high calorie content. Protein foams, like meringue made from egg whites, typically have a less silky mouth feel, are destabilized by lipids in the formation phase, and contain significantly fewer calories than fat based foams when compared with no added sweeteners.

Foam stability is critical to oral processing duration and gastric distension, key factors in satiation. Foam stability also provides a textural component known to food formulators as mouth feel, a determinant of consumer satisfaction, and a possible cue in sensory specific satiation (i.e., creamy textures provide more satiation than watery textures). Without some degree of foam stability, the semisolid will return to its liquid form before it can be ingested and enjoyed by the consumer. Preferred embodiments would retain most of their foam structure for at least 30 seconds once expressed from the aerosol delivery system. For purposes of this application, foam is determined to be adequately stable if 70% of the foam volume remains after 30 seconds of being dispensed.

As an integral part of the volume expansion and subsequent consumer benefit, food emulsions used in these embodiments should have the chemical properties necessary to produce stable foam. Since food foams will contain either protein, fat or a combination of the two, we'll discuss three general variations: 1) enhanced protein, virtually fat free; 2) Mixed fat and protein; 3) high fat, various protein levels.
1. When whipped or otherwise agitated, proteins align themselves between air and water to form bubbles, and bond to one another side-to-side as crosslinks which add to the protein foam's stability. Fat molecules also have both hydrophobic and hydrophilic parts and will compete with proteins in the hydrophobic/hydrophilic environment. Instead of reinforcing the protein network, fats compete with protein molecules in forming bonds, thereby destabilizing the foaming system. In short, the presence of even relatively small amounts of fat during the foaming stage impedes formation and/or breaks down protein foams quickly. Therefore, for protein foam to have stability after dispensing, protein based foam, one embodiment, must be free of as much fat as possible, preferably below 1% of total weight.
2. Emulsions comprised of a mixture of protein and fat have varying degrees of stability, with some stable bubble formation occurring once fat content reaches about 7 percent with stabilization improving as fat content increases. It may be advantageous to add ingredients containing good fats to some embodiments (e.g., omega 3, cocoa, etc.). Fats are high in calories and generally lower in satiation, so the benefits of adding fat to the emulsion need to be weighed against the additional calories and the purpose of the embodiment. Food stabilizing systems, including gums, lecithin, gelatin, agar and others help increase foam stability of protein-based, fat-based and combination foams. Food scientists are currently working to create protein foaming systems that are resistant to the presence of lipids which would provide more flexibility with regard to quantity of fat used and calorie content. When those systems become available in the marketplace, one or more embodiments will include them in emulsion design if they offer advantages over current technology.
3. Recent research indicates that diets high in good fats and low in carbohydrates may be preferable for those suffering from type 2 diabetes, as the high fat diet has shown to help in the regulation of blood sugar. It's important to stress that milk fat or hydrogenated oils (trans fats) traditionally used to make edible fat foams are not good alternatives as both are extremely high in saturated fat, a potential risk factor for those suffering from diabetes. As scientific research provides greater insight into nutritional therapies for chronic disease, varying embodiments' nutritional profiles can be tailored to meet the dietary needs of diabetics and those suffering from other diet responsive diseases.

Varying protein, carbohydrate and fat combinations provide for myriad embodiments based on designer emulsions tailored for many aims, including but not limited to chronic—diet responsive—disease management, weight loss, weight management, athletic performance, improved muscle mass, energy management, nutritional wellness and many others. As drug and nutritional therapies advance, they may be incorporated into additional embodiments as well.

One embodiment is intended to deliver a low calorie semisolid for purposes of calorie reduction and weight loss. Given that protein has excellent foaming qualities and studies suggest that it provides higher satiation than fat, this embodiment will use protein as the main foaming ingredient.

Referring now to the invention in further detail, in accordance with one embodiment, 400 grams of a food emulsion with the following ingredients is filled into a disposable aerosol can known to those skilled in the art of aerosol whipped topping manufacture and charged with a propellant, nitrous oxide gas:

Emulsion Formulation by Weight -

| | |
|---|---|
| Non fat milk | 84% |
| Cane sugar | 11% |
| Whey protein concentrate | 3% |
| Natural flavor vanilla | .963% |
| Vitamin/Mineral/Fiber nutritional blend | 1% |
| Carrageenan | .037% |
| Total | 100% |

The emulsion will have the chemical properties to foam when dispensed from an aerosol delivery system with an expansion of volume (overrun) of between 250% and 400%. The aerosol delivery system in this embodiment is a disposable metal can with plastic nozzle dispensing mechanism and cap known to those familiar in the art of aerosol whipped topping manufacture, and will be charged with nitrous oxide gas in an amount sufficient to promote overrun of at least 300%.

The Invention is not limited by the current state of the art in aerosol container manufacturing. New and innovative aerosol packaging is in development (e.g., plastic containers, new nozzle mechanism designs, new propellants, aseptic aerosol containers requiring no refrigeration, etc.). Future embodiments of the Invention will utilize these developments should they demonstrate advantages over current technologies.

The Invention is novel and embodiments should not to be confused with condiments like aerosol whipped topping or aerosol cheese spread that are currently available in grocery stores. Aerosol whipped topping is primarily a compliment to other products like ice cream or strawberry short cake. In the same way, aerosol cheese spreads are compliments to crackers and chips. Aerosol whipped toppings and aerosol cheese spreads are not generally consumed as stand-alone products and are not recommended to be consumed in large quantities due to health concerns related to their high calorie load and saturated fat content. Nor are these toppings/spreads recommended to be consumed directly from the package as they are stored often for months allowing bacteria to multiply on the dispensing mechanism, thereby contaminating the food when dispensed. Neither aerosol whipped toppings nor aerosol cheese spreads are fortified with nutrients that would allow them to be good candidates for meal replacements, diet shakes, or healthy “stand alone” snacks.

The FDA established Reference Amount Customarily Consumed (RACC) for 139 food product categories, and these values represent the amount of food customarily consumed at one eating occasion. These appear as the Serving Size on the Nutrition Statement incorporated into most retail food packaging. The serving size for both aerosol whipped topping and aerosol cheese spread is 2 tablespoons. By comparison, one embodiment as a meal replacement has a serving size of 8 fluid ounces before expressing and 24 fluid ounces following delivery from the aerosol package. The RACC does not currently have a category that addresses the consumption volumes intended by one or more embodiments of the invention following delivery from the aerosol delivery system. As it is the responsibility of the inventor/manufacturer of “out of category” food products to request a new RACC category ruling from the FDA, the fact that no request has been made to the FDA is further evidence of the uniqueness of the invention.

The advantages of the present invention include, without limitation,

### Advantages—Weight Loss/Weight Maintenance

Satiation happens in the brain. The stomach does not have a calorie counter, per se. Due to the time lag between consumption of food and its subsequent digestion, the body needs a short term system to help with appetite regulation. Although researchers have not yet unraveled the complete mystery of satiation and appetite regulation, recent studies suggest that, as part of a complex process, satiation information is conveyed to the brain in a number of ways that some researchers call the satiation feedback loop. This feedback is both physiologically and psychologically derived, and some aspects are discussed below:

Calorie Content: According to recent studies, all foods are not equal when it comes to satiation. The Satiation Index, created by Dr. Susanne Holt at the University of Sydney, suggests that foods high in protein offer more satiation than those high in carbohydrates and fats. One embodiment of the invention utilizes the Satiation Index among other scientifically-based nutritional models to provide a healthy emulsion that can be used to reduce hunger cravings or as a full meal replacement.

Heightened Oral Processing: Thoroughly chewing your food increases the stimulation of oro-sensory receptors in the lips, tongue, palette and olfactory system. Signals are transmitted from these receptors in the oral cavity to the olfactory bulb and are interpreted by the brain as part of the appetite regulation process. Studies suggest that the process of masticating distributes flavors, textures and scents throughout the mouth and olfactory system far more effectively than drinking. The more effective distribution contributes to greater oro-sensory exposure which is correlated with decreased food intake. Numerous studies conclude that eating rather than drinking provides greater satiation, even when the calories consumed are the same. A principal advantage of the Invention over some prior art (namely, weight loss shakes, smoothies and beverages) is that by transforming a liquid into a semisolid, consumption of the semisolid requires greater oral processing than it would in its liquid state, thereby increasing both contact area in the oral cavity (lips, tongue, pallet, olfactory sensors) and duration of contact. Studies conclude that this increase in duration and contact increases satiation. Gastric (Stomach) Distention: Various studies have used mechanical methods to simulate gastric distention with both human and animal subjects. These studies have concluded that gastric distention likely triggers satiation signals and influences the quantity of food consumed. In one embodiment, the volume of the food emulsion is increased by 300% as a result of being dispensed from the aerosol delivery system. The emulsion's increased volume fills and distends the stomach, thereby eliciting a gastric distention satiation response. By comparison, the per calorie volume of weight loss shakes, smoothies and beverages is significantly less and, therefore, provide less satiation from gastric distention.

Slow Eating Increases Satiation: Recent studies confirm the popular belief that eating more slowly increases satiation as compared to eating more quickly. Studies suggest that oro-sensory exposure time has a meaningful effect on food intake and sensory specific satiation. A main disadvantage of weight loss shakes/drinks is the speed with which they are consumed (drinking vs. eating). Many studies indicate that slower consumption equates to higher satiation. By transforming a liquid food emulsion into a semisolid with an increased volume of 300% as with one embodiment, the consumption process is slowed significantly and satiation is thereby increased beyond that of current weight loss drinks, shakes or smoothies.

Learned Satiation: Recent studies suggest that a person's expectation that thicker foods provide more satiation will, in turn, influence the degree to which that person feels satiated. This is called Learned Satiation. This is one reason that weight loss beverages frequently add thickeners to promote a more viscous quality especially when blended or shaken. The blending/shaking process imparts gas into the thickened emulsion which adds volume. The Invention is far more effective at adding volume, and physically transforms the emulsion into a semisolid, which is significantly more viscous than liquid weight loss shakes, smoothies and beverages. As a result, the consumer will expect more satiation from the Invention and is therefore more likely to derive psychological satiation when compared to thinner weight loss shakes, smoothies and beverages. Comparison of Volume between prior art and one embodiment of the invention: Most weight loss shakes suggest a serving size of 8 fluid ounces. By comparison, an equivalent 8 fluid ounces of emulsion, the ingredients of which have been previously listed, increases to 24 fluid ounces or more of semisolid once dispensed from one embodiment, a disposable aerosol can familiar to those skilled in the art of aerosol whipped topping manufacture. As compared to a typical weight loss shake, this embodiment produces a net improvement of 300% in volume, despite having similar nutritional and caloric value. This additional volume influences several of the satiation feedback mechanisms previously discussed.

Convenience: Weight loss shakes, smoothies and beverages have varying degrees of convenience from opening a ready-to-drink container (typically exhibiting a small amount of additional volume from shaking or manual agitation) to liquids and rehydrates that require mixing in blenders (typically gaining more volume through gas induction and foaming than shaking or manually agitating alone, but far less than the volume produced by embodiments of the Invention). Convenience is a factor in any weight management program since competitive influences, like processed foods and fast food restaurants, specialize in easily available, quick, highly tempting foods. A significant advantage of the invention is that multiple servings can be packed into a convenient aerosol container that can be dispensed directly into the mouth. Part of the convenience is simply the volume of food contained in the Invention. Consider that a ready-to-drink weight loss shake may provide 8 fluid ounces and 180-240 calories, as compared to a similar or slightly larger sized package—one embodiment of the invention—that provides approximately 24 fluid ounces of a semisolid with approximately the same calorie load. One potential embodiment, a larger-sized aerosol whipped topping package commonly found on grocery shelves, can produce 48 or more fluid ounces of semisolid. To ingest a comparable volume of a ready-to-drink diet shake would require 6 eight ounce bottles.

Consider that calorie loads in varying embodiments can be adjusted to correspond with desired servings per container, allowing flexibility for multiple aims.

### Advantages—Chronic Disease Management

Because many diet responsive diseases like Type 2 Diabetes are exacerbated, or even caused, by being overweight, a key advantage of the invention is its flexibility with regard to emulsion design. Because the semisolid product of the invention can be tailored nutritionally to address multiple health issues, varied embodiments can be developed to address myriad diet-related health concerns.

Because consumers have varying dietary needs to maintain or improve health, embodiments will offer emulsions specifically formulated to promote healthy weight loss or weight management for specific subgroups. For instance, patients recovering from gastric bypass surgery cannot, in many cases, eat solid food for several months following surgery. They also have to compensate nutritionally for reduced food consumption, while avoiding certain foods that may cause dumping syndrome. Therefore, one embodiment is rich in protein, highly fortified with vitamins and nutrients, and is low in refined sugars and other ingredients that may aggravate or promote dumping syndrome.

With regard to diabetes, one embodiment reduces carbohydrates and enhances fiber to slow the absorption of carbohydrates after consumption. As previously mentioned, a diet high in good fats and low in carbohydrates may help diabetics better control their blood sugar. In this connection, one embodiment is formulated to deliver a moderate to high fat (polyunsaturated & monounsaturated fats, low in saturated fat), moderate to high protein, low carbohydrate semisolid.

In broad embodiment, the invention is a method of transforming a food emulsion into a semisolid and delivering said semisolid to a person in order to further his/her weight loss or weight management aims by stimulating satiation feedback mechanisms.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

### CITATIONS

R D Mattes, W W Campbell, “Effects of food form and timing of ingestion on appetite and energy intake in lean young adults and in young adults with obesity,” Journal of American Diet Association, March 2009; 109(3):430-7. doi: 10.1016/j.jada.2008.11.031
S. H. A. Holt, J. C. Brand Miller, P. Petocz, and E. Farmakalidis, “A Satiety Index of Common Foods,” European Journal of Clinical Nutrition, September 1995, pages 675-690
Nicolien Zijlstra, René de Wijk, Monica Mars, Annette Stafleu, and Cees de Graaf, “Effect of bite size and oral processing time of a semisolid food on satiation,” The American Journal of Clinical Nutrition, Jun. 10, 2009, doi: 10.3945/ajcn.2009.27694 Am J Clin Nutr August 2009 vol. 90 no. 2 269-275
Martin R Yeomans and Lucy Chambers, “Satiety-relevant sensory qualities enhance the satiating effects of mixed carbohydrate-protein preloads,” The American Journal of Clinical Nutrition, Oct. 26, 2011, doi: 10.3945/ajcn.111.011650 Am J Clin Nutr December 2011 vol. 94 no. 6 1410-1417
Jie Li, Na Zhang, Lizhen Hu, Ze Li, Rui Li, Cong Li, and Shuran Wang, “Improvement in chewing activity reduces energy intake in one meal and modulates plasma gut hormone concentrations in obese and lean young Chinese men,” The American Journal of Clinical Nutrition, Jul. 20, 2011, doi: 10.3945/ajcn.111.015164 Am J Clin Nutr September 2011 vol. 94 no. 3 709-716
R. M. Ruijschop, A. E. M. Boelrijk, M. J. M. Burgering, C. de Graaf, and M. S. Westerterp-Plantenga, “Novel Approaches to Induce Satiation Via Aroma in Foods,” Expression of Multidisciplinary Flavour Science, 2010, pages 41-44
Pascalle L. G. Weijzen, Paul A. M. Smeets and Cees de Graaf, “Sip size of orangeade: effects on intake and sensory-specific satiation,” British Journal of Nutrition (2009), 102, 1091-1097, doi:10.1017/S000711450932574X
Mojtahedi M C, Thorpe M P, Karampinos D C, Johnson C L, Layman D K, Georgiadis J G, Evans E M., “The effects of a higher protein intake during energy restriction on changes in body composition and physical function in older women,” J Gerontol A Biol Sci Med Sci. 2011 November; 66(11):1218-25. Epub 2011 Jul. 27.
Bray G A, Smith S R, de Jonge L, et al. “Effect of Dietary Protein Content on Weight Gain, Energy Expenditure, and Body Composition During Overeating—A Randomized Controlled Trial,” Journal of the American Medical Association, 2012; 307(1):47-55. doi: 10.1001/jama.2011.1918
Fowler S P, Williams K, Resendez R G, Hunt K J, Hazuda H P, Stern M P.,“Fueling the obesity epidemic? Artificially sweetened beverage use and long-term weight gain.,” Obesity (Silver Spring), 2008 August; 16(8):1894-900.
S. N. Mayer, R. S. Davidson and C. B. Hensley, “The Role of Specific Olfactory Stimulation in Appetite Suppression and Weight Loss,” Journal of Advancement in Medicine, Volume 12, Number 1 (1999), 13-21.
Emma R Dove, Jonathan M Hodgson, Ian B Puddey, Lawrence J Beilin, Ya P Lee, Trevor A Mori, “Skim milk compared with a fruit drink acutely reduces appetite and energy intake in overweight men and women,” Am J Clin Nutr July 2009 90: 1 70-75

The embodiments of the invention in which an exclusive property or privilege is claimed are defined as follows:

1. A method of enhancing weight loss and/or weight maintenance in a person, comprising the transformation of a food emulsion into a semisolid, through use of an aerosol delivery system, and providing said semisolid to said person for ingestion.

2. The method of claim 1, wherein said semisolid can be dispensed directly into said person's mouth from said aerosol delivery system, avoiding the need for eating utensils, and with limited potential for bacterial contamination when the contents are consumed in one sitting.

3. The method of claim 1, wherein said semisolid can be dispensed directly into said person's mouth from said aerosol delivery system, avoiding the need for eating utensils, and with limited potential for bacterial contamination when the nozzle is cleansed with mild detergent and rinsed with water after contact with the mouth or other source of contamination.

4. The method of claim 1, wherein weight loss and/or weight maintenance is enhanced, further comprising the transformation of a food emulsion, which has been nutritionally, functionally and calorically tailored to aid in healthy weight loss or weight maintenance, into a semisolid through use of said aerosol delivery system, and providing said semisolid to said person for ingestion.

5. A method of claim 1, whereby a person recovering from weight loss surgery is aided in weight loss and/or weight maintenance, further comprising the transformation of a food emulsion, nutritionally, functionally and calorically tailored for the specific dietary needs of weight loss surgery aftercare patients, into a semisolid through use of said aerosol delivery system and providing said semisolid to said person for ingestion.

6. A method of claim 1, wherein persons with diet responsive diseases like type 2 diabetes are aided in weight loss and/or weight maintenance, further comprising the transformation of food emulsions, nutritionally, functionally and calorically tailored to the best available dietary, scientific and medical recommendations, relative to an array of specific diet responsive diseases, into semisolids through use of said aerosol delivery system, and providing said semisolids to said persons for ingestion.

7. The method of claim 4, wherein weight loss and/or weight maintenance is enhanced, further comprising the transformation of a food emulsion, which has been nutritionally, functionally and calorically tailored to be a healthy meal replacement, into a semisolid through use of said aerosol delivery system and providing said semisolid to said person for ingestion as a replacement for one or two meals on an ongoing basis, or as a replacement for all daily meals on a temporary basis when combined with proper medical supervision.

8. The method of claim 4, wherein said semisolid provides said person with a substitute for between meal snacks, or is ingested by said person immediately preceding meals, thereby stimulating satiation feedback mechanisms to aid in hunger regulation.

9. The method of claim 1, wherein an aerosol delivery system consists of a propellant, such as nitrous oxide gas, and a food emulsion, both, of which are filled, observing the appropriate sequence, into a commercially available, disposable aerosol package, typically a metal aerosol can with plastic dispensing nozzle mechanism and cap, known to those skilled in the art of aerosol whipped topping manufacture, aseptic or otherwise.

10. The method of claim 9, wherein the aerosol delivery system is charged with a commercially available propellant, such as nitrous oxide gas, in an amount sufficient to create stable foam when dispensed.

11. The method of claim 1, wherein an aerosol delivery system consists of a propellant, such as nitrous oxide gas, and a food emulsion, both of which are filled, observing the appropriate sequence, into a reusable canister commonly available for home and commercial use for the purpose of converting emulsions like heavy whipping cream into aerosol whipped cream.

12. The method of claim 11, wherein a dehydrated food emulsion with rehydrating liquid or a prepackaged liquid food emulsion is filled into a reusable pressurized canister, which is then charged with a propellant for the purposes of transforming said emulsion into stable foam.

13. The method of claim 2, wherein a person dispenses said semisolid from said aerosol delivery system directly into said person's mouth by inverting said aerosol delivery system so that its bottom is held at least 10 degrees above the dispensing nozzle mechanism.

14. The method of claim 3, wherein said person dispenses said semisolid from said aerosol delivery system directly into said person's mouth by inverting said aerosol delivery system so that its bottom is held at least 10 degrees above the dispensing nozzle mechanism.
